(19) United States
(12) Patent Application Publication
(10) Pub. No.: US 2016/0220412 A1
(43) Pub. Date: Aug. 4, 2016

(54) CONDOM

(71) Applicant: Alex Vacco, Douglas, MI (US)

(72) Inventor: Alex Vacco, Douglas, MI (US)

(21) Appl. No.: 14/884,825

(22) Filed: Oct. 16, 2015

Related U.S. Application Data
(60) Provisional application No. 62/109,905, filed on Jan. 30, 2015.

(51) Int. Cl. A61F 6/04

(52) U.S. Cl. CPC A61F 6/04; A61F 2006/048

(57) ABSTRACT

A condom is disclosed having a foraminous side wall having a plurality of openings for enhancing sensation and encouraging usage.

### CROSS REFERENCE TO RELATED APPLICATIONS

This application claims the benefit of U.S. Provisional Application No. 62/109,905 filed on Jan. 30, 2015. The patent application identified above is incorporated here by reference in its entirety to provide continuity of disclosure.

### TECHNICAL FIELD

This disclosure relates to condoms for contraceptive and prophylactic use, and more particularly to improved condoms for enhanced pleasure.

### BACKGROUND

The statements in this section merely provide background information related to the present disclosure and may not constitute prior art.

Condoms are well known in the art. Condoms have a dual use as a contraceptive, to inhibit or prevent semen from being deposited in a woman's cervix, and as a prophylactic to inhibit or prevent sexually transmitted infections (STI's) such as the HIV virus, which causes the Auto-Immune Deficiency Syndrome (AIDS). Most modern condoms are made from latex.

The use of condoms inhibits the spread of STI's. Considerable, world-wide effort has been made to promote the adoption of condoms to prevent infection and unwanted pregnancies. The use of condoms as a contraceptive method and as a method of preventing the spread of STI's has is viewed as urgent, both from a medical and a societal standpoint. Unfortunately, the widespread adoption of condom use (both in the US and internationally) has been limited by a number of factors. From the male user's perspective, condoms inhibit sensation and pleasure due to the interposition of the condom between the penis and the vagina during intercourse. This drawback causes condoms to be inconsistently or intermittently used.

Therefore, there is a need for an improved condom that enhances sensation while still functioning as a contraceptive and prophylactic device.

### SUMMARY

An elastomeric condom, including a closed distal end portion to cover the glans of the penis, said closed end portion terminating in a teat for the collection of semen, an axial hollow tubular portion to cover a shaft of the penis having an inner surface and an outer surface, and having an open proximal end, wherein the hollow tubular portion includes a discontinuous portion having a plurality of openings.

A condom is disclosed having a foraminous, i.e., discontinuous side wall for enhancing sensation and encouraging usage. The side wall may be foraminous or discontinuous by virtue of a plurality of openings, the openings having a particular shape type from any number of shape types in various embodiments including hexagon, octagon, elliptical, rectangular, circular, square, and/or diamond.

Various embodiments include bands formed axially or circumferentially of the condom sheath and having less flexibility than the body of the axial tubular sheath so as to aid in keeping the condom in place during use. One or more of the bands may be positioned between the closed end and the openings.

Various embodiments include an outwardly extending protrusion integrally formed of the condom sheath around one or more of the openings.

This summary is provided merely to introduce certain concepts and not to identify key or essential features of the claimed subject matter.

### BRIEF DESCRIPTION OF THE DRAWINGS

One or more embodiments will now be described, by way of example, with reference to the accompanying drawings, in which:

FIG. 1 is a perspective view of an exemplary condom, in accordance with the present disclosure;

FIG. 2 is a front view of the exemplary condom, in accordance with the present disclosure;

FIG. 3 is a cross-sectional view of the exemplary condom taken along line A-A of FIG. 2, in accordance with the present disclosure;

FIG. 4 is a cross-sectional view of the exemplary condom taken along line B-B of FIG. 2, in accordance with the present disclosure;

FIGS. 5A and 5B show an exemplary embodiment of the condom having hexagonal openings, in accordance with the present disclosure;

FIGS. 6A and 6B show an exemplary embodiment of the condom having hexagonal openings with additional rows, in accordance with the present disclosure;

FIGS. 7A and 7B show an exemplary embodiment of the condom having rectangular openings, in accordance with the present disclosure;

FIGS. 8A and 8B show an exemplary embodiment of the condom having octagonal openings, in accordance with the present disclosure; and

FIGS. 9A and 9B show an exemplary embodiment of the condom having elliptical or circular openings, in accordance with the present disclosure.

### DETAILED DESCRIPTION

Throughout the specification and claims, the following terms take at least the meanings explicitly associated herein, unless the context dictates otherwise. The meanings identified below do not necessarily limit the terms, but merely provide illustrative examples for the terms. The meaning of “a,” “an,” and “the” includes plural reference, and the meaning of “in” includes “in” and “on.” The phrase “in one embodiment,” as used herein does not necessarily refer to the same embodiment, although it may. Similarly, the phrase “in various embodiments,” as used herein, when used multiple times, does not necessarily refer to the same embodiments, although it may. As used herein, the term “or” is an inclusive “or” operator, and is equivalent to the term “and/or,” unless the context clearly dictates otherwise. The word “exemplary” is used herein to mean “serving as an example, instance, or illustration.” Any aspect described herein as “exemplary” is not to be construed as exclusive, preferred or advantageous over other aspects.

Various embodiments of the present invention will be described in detail with reference to the drawings, where like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

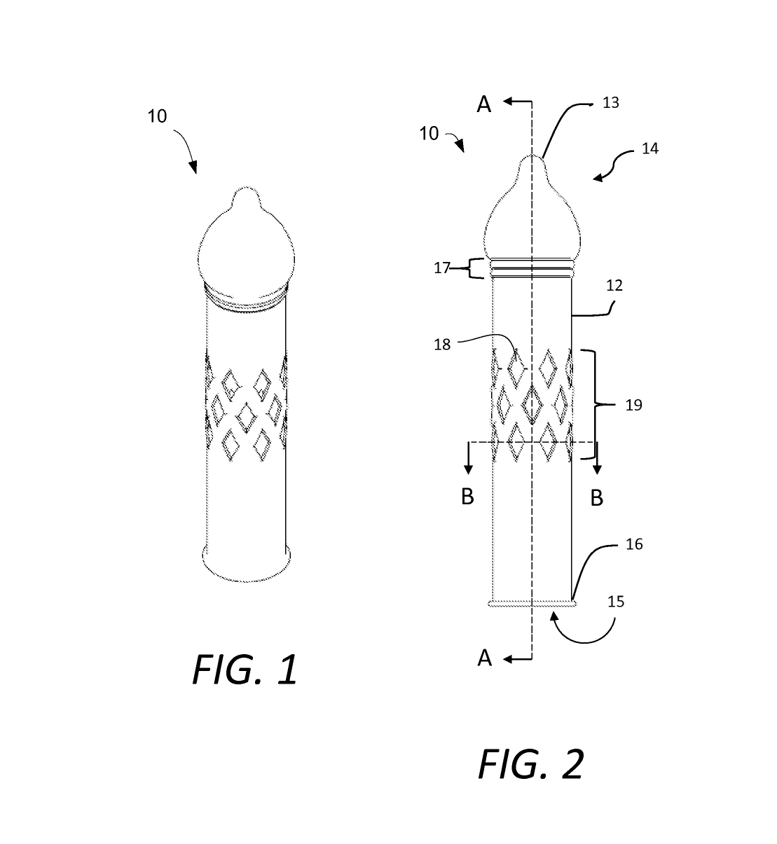

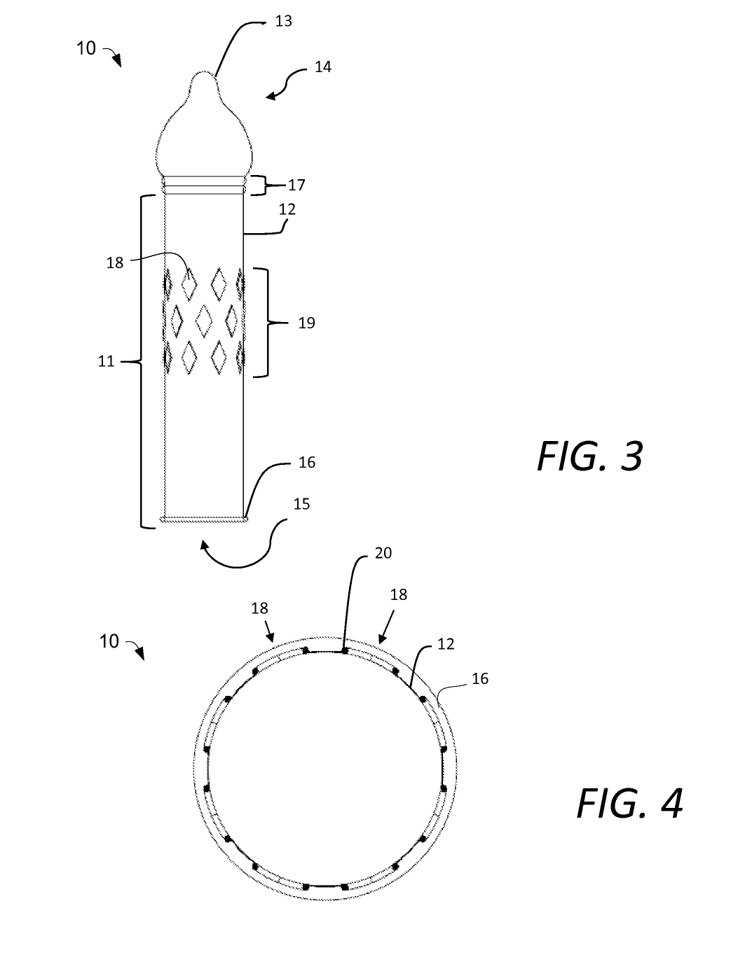

Referring now to the drawings, wherein the depictions are for the purpose of illustrating certain exemplary embodiments only and not for the purpose of limiting the same, FIGS. 1-4 show a first embodiment of a condom 10 as one embodiment the present invention in the unfurled, unrolled condition.

The condom 10 is flexible and is preferably is made of a resilient material, such as latex or various synthetic polymeric materials, e.g., thermoplastic elastomeric material. The condom 10 is preferably formed of substantially uniform thickness. Upon a careful reading of the teachings herein, one skilled in the art will readily recognize that the disclosure can be applied to various resilient materials and is therefore not intended to be limited thereby including any material commonly used for the manufacture of condoms or that may be suitable for such usage. For example, the condom and the elastic material from whence it is formed can be of material having an elastic impermeable characteristic once shaped and removed from the mold. Examples of such materials include substances such as natural rubber (e.g. latex); synthetic rubber (e.g. silicone rubber); polyurethane; and other elastic substances such as thermoplastic polyolefin elastomers.

The exemplary condom 10 is formed of sheath 12 having a distal closed end portion 14 and a proximate open end 15. The open end 15 preferably includes a reinforced rib or lower band 16 to provide friction, holding the condom 10 in place during use. The lower band 16 is shaped as a generally circular torus and is made from the same material as the sheath 12 positioned at an axial end 15 of sheath 12. The lower band 16 surrounds the opening 15. The lower band 16 may be formed as an integral part of the sheath 12. In one embodiment, the distal closed end portion 14 is an enlarged portion of the condom 10 having a circumference and diameter slightly larger than associated with the lower portion 11 of the sheath 12.

In one embodiment, the condom 10 includes one or more upper bands 17 to hold the distal closed end 14 in place during use. The one or more upper bands 17 are generally circular torus-shaped and may be made from the same material as the sheath 12. In one embodiment, the bands 17 are formed as an integral part of the sheath 12.

The sheath 12 is a generally cylindrical or elongated tubular shaped structure. In various embodiments, the sheath 12 may have a semen receptacle 13 integrally formed thereof at a distal end. In various embodiments, lubricant and various agents may be deposited on interior and exterior surface portions of the sheath 12. These agents can include spermicides and microbicides which inactivate or block infection by sexually transmitted pathogens.

The condom 10 includes a plurality of openings 18 forming a discontinuous portion 19 of the sheath 12. The discontinuous portion 19, in one embodiment, is positioned in an upper to central area of the condom 10. In one embodiment, the openings 18 are generally diamond or rhombus-shaped and are uniformly arranged in rows and columns. In one embodiment, the rows with openings 18 arranged under each other may be each offset essentially from each other by the half-width of an opening. In one embodiment, columns with openings 18 arranged adjacent each other may be offset laterally from each other, such as shown in FIG. 1.

FIG. 4 shows a cross-sectional view of the condom 10 along line B-B of FIG. 2. As FIG. 4 shows, the openings 18, in one embodiment, are generally bordered by a raised protrusion 20 formed around the openings 18. The raised protrusion 20 forms a perimeter around the openings 18 and extends outwardly from an outer surface of the sheath 12. In various embodiments, the raised protrusion 20 may extend outwardly from the sheath 12 and inwardly from the sheath 12.

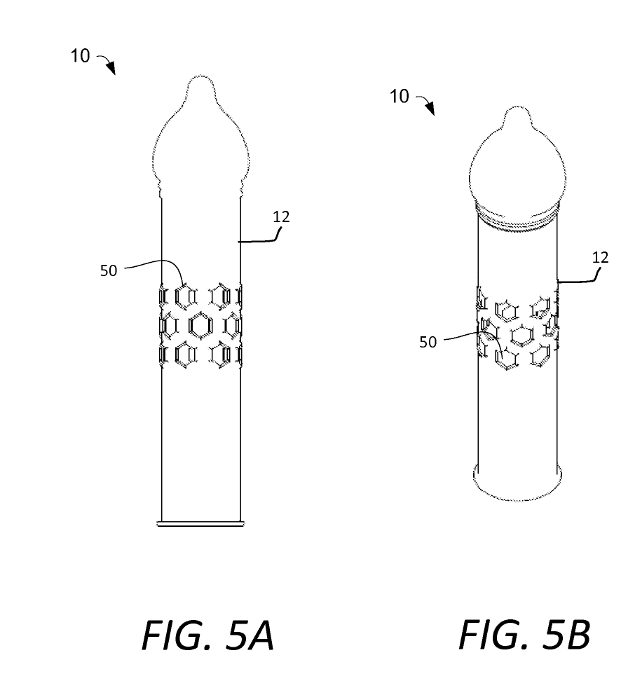

FIGS. 5A-9B show various embodiments of the openings of the condom 10 including hexagon shapes, square shapes, circular shapes, and octagonal shapes. In various embodiments, it is contemplated that the arrangement, quantity of openings, and size of openings may vary.

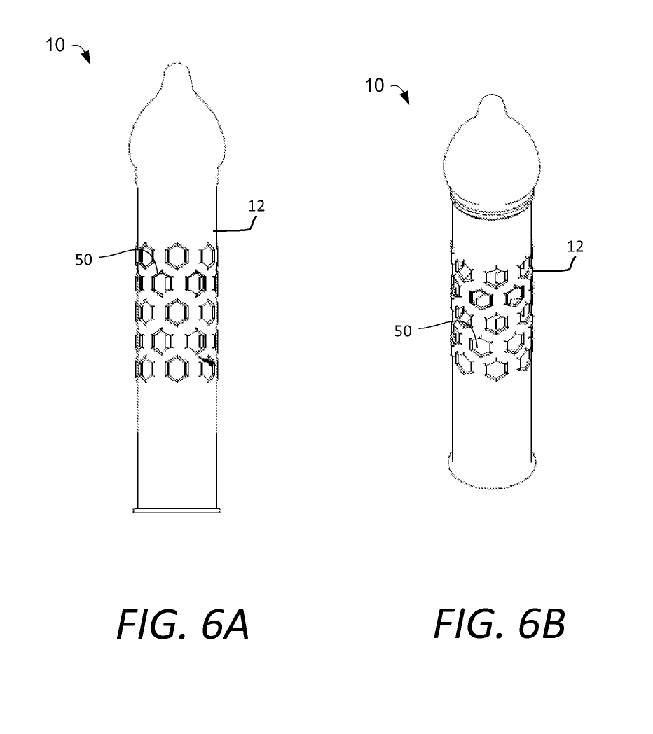

FIGS. 5A and 5B and FIGS. 6A and 6B show an embodiment of the condom 10 having hexagonal shaped openings 50 formed to a region of the sheath 12. FIGS. 5A and 5B show the hexagonal shaped openings as arranged in three rows, while FIGS. 6A and 6B show the hexagonal shaped openings as arranged in five rows. In one embodiment, the region is positioned in a central area of the condom 10. The openings 50 are generally hexagonal shaped and are uniformly arranged in rows and columns, such that a generally honeycomb structure is produced. In one embodiment, the rows with openings 50 arranged under each other may be each offset essentially from each other by the half-width of an opening. In one embodiment, columns with openings 50 arranged adjacent each other may be offset laterally from each other, such as shown in best in FIG. 5A and FIG. 6A.

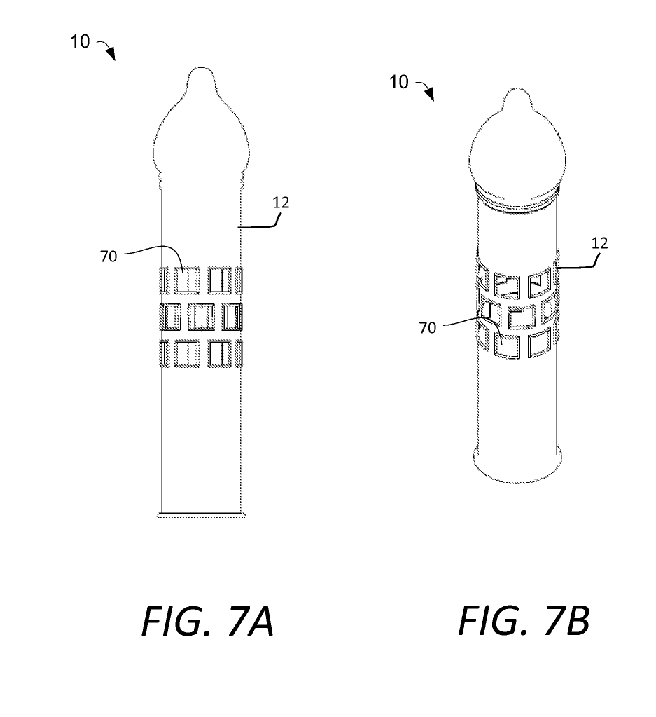

FIGS. 7A and 7B shows an embodiment of the condom 10 having square shaped-openings 70 formed to a region of the sheath 12. In one embodiment, the region is positioned in a central area of the condom 10. The openings 70 may be generally oblong shaped-openings such as the square shapes shown, but may be rectangular and are preferably uniformly arranged in rows and columns. In one embodiment, the rows with openings 70 arranged under each other may be each offset essentially from each other by the half-width of an opening. In one embodiment, columns with openings 70 arranged adjacent each other may be offset laterally from each other, such as shown in FIGS. 7A and 7B.

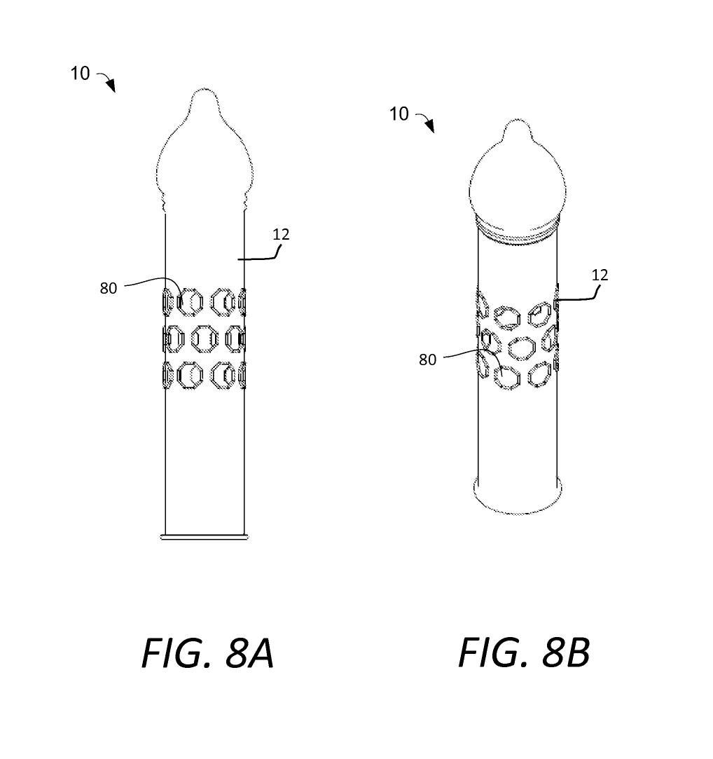

FIGS. 8A and 8B shows an embodiment of the condom 10 having octagonal shaped-openings 80 formed to a region of the sheath 12. In one embodiment, the region is positioned in a central area of the condom 10. The openings 80 may be generally octagonal and are preferably uniformly arranged in rows and columns. In one embodiment, the rows with openings 80 arranged under each other may be each offset essentially from each other by the half-width of an opening. In one embodiment, columns with openings 80 arranged adjacent each other may be offset laterally from each other, such as shown in FIGS. 8A and 8B.

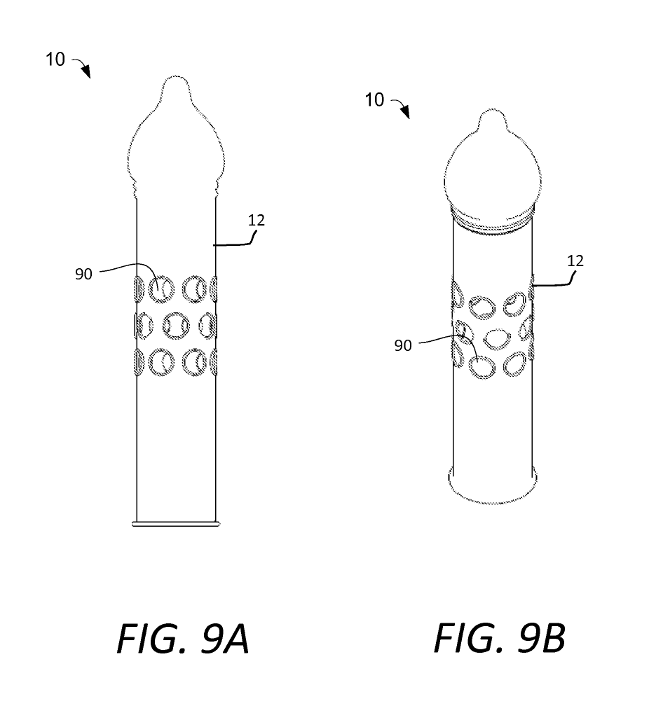

FIGS. 9A and 9B shows an embodiment of the condom 10 having circular shaped-openings 90 formed to a region of the sheath 12. In one embodiment, the region is positioned in a central area of the condom 10. The openings 90 are generally circular or elliptical-shaped and are uniformly arranged in rows and columns. In one embodiment, the rows with openings 90 arranged under each other may be each offset essentially from each other by the half-width of an opening. In one embodiment, columns with openings 90 arranged adjacent each other may be offset laterally from each other, such as shown in FIGS. 9A and 9B.

Other suitable shapes are conceivable that facilitate a large area for the openings combined with simultaneous stability of the interlaying framework.

With respect to the mutual arrangement of the openings, these may either be arranged in rows, in which the openings are entirely spaced apart in rows, or the openings are arranged in the rows such that they project into the corresponding cavities formed by openings of adjacent rows. The openings in the columns in which they are arranged may be provided directly beneath each other or offset from each other, such that load dissipation, improves. The columns with openings arranged under each other may be each offset essentially from each other by the half-width of an opening, in one embodiment.

The disclosure has described certain preferred embodiments and modifications thereto. Further modifications and alterations may occur to others upon reading and understanding the specification. Therefore, it is intended that the disclosure not be limited to the particular embodiment(s) disclosed for carrying out this disclosure, but that the disclosure will include all embodiments falling within the scope of the appended claims.

1. An elastomeric condom, comprising:
a closed distal end portion to cover the glans of the penis, said closed end portion terminating in a teat for the collection of semen;
an axial hollow tubular portion to cover a shaft of the penis having an inner surface and an outer surface, and having an open proximal end, wherein the hollow tubular portion includes a discontinuous portion having a plurality of openings.

2. The condom of claim 1, further comprising:
a band circumferentially, integrally formed of the axial hollow tubular portion and having less elasticity than the axial hollow tubular portion.

3. The condom of claim 2, wherein the band is disposed between the distal end portion and the discontinuous portion.

4. The condom of claim 1, wherein the discontinuous portion is centrally located on the tubular portion.

5. The condom of claim 1, wherein the plurality of openings are arranged in circumferential rows between the distal closed end and the open proximal end.

6. The condom of claim 5, wherein the rows are offset from each other.

7. The condom of claim 5, wherein the rows are offset from each other by a half-width of an opening plus a half-width of a space between openings.

8. The condom of claim 1, wherein the openings are hexagonal shaped.

9. The condom of claim 1, wherein the openings are elliptical shaped.

10. The condom of claim 1, wherein the openings are oblong shaped.

11. The condom of claim 1, wherein the openings are octagonal shaped.

12. The condom of claim 1, wherein the openings are diamond shaped.

13. A condom, comprising:
an elongated thin-walled tubular sheath of resilient material having a distal closed end and an open proximal end, wherein the sheath comprises a foraminous side wall of discrete openings arranged in circumferential rows between the distal closed end and the open proximal end, wherein the discrete openings are perpendicular to the open proximal end; and
a protrusion integrally formed of the tubular sheath around one or more of the openings, the protrusion outwardly extending from an outer surface of the tubular sheath.

14. The condom of claim 13, wherein the foraminous side wall is centrally located on the tubular portion.

15. The condom of claim 13, further comprising:
a band circumferentially, integrally formed of the axial hollow tubular portion and having less elasticity than the axial hollow tubular portion.

16. The condom of claim 15, wherein the band is disposed between the distal end portion and the discontinuous portion.

17. The condom of claim 13, wherein the rows are offset from each other by a half-width of an opening plus a half-width of a space between openings.

18. The condom of claim 13, wherein the openings are hexagonal shaped.

19. The condom of claim 13, wherein the openings are diamond shaped.

20. A condom, comprising:
an axial tubular sheath formed of flexible polymeric material, said condom comprising a body having a closed end and an open end;
a plurality of discrete openings arranged axially along the tubular sheath in rows, the discrete openings positioned between the distal closed end and the open proximal end of the axial tubular sheath, wherein the discrete openings are perpendicular to the open proximal end, and wherein the circumferential rows are offset from one another by at least a half-width of one of the openings;
a band circumferentially, integrally formed of the axial hollow tubular portion and having less flexibility than the body of the axial tubular sheath; and
an outwardly extending protrusion integrally formed of the axial tubular sheath around one or more of the openings.
